(19) United States
(12) Patent Application Publication
(10) Pub. No.: US 2019/0066845 A1
(43) Pub. Date: Feb. 28, 2019

(54) DISTRIBUTED ANALYTICS SYSTEM FOR IDENTIFICATION OF DISEASES AND INJURIES

(71) Applicant: The Charles Stark Draper Laboratory, Inc., Cambridge, MA (US)

(72) Inventors: Nilay K. Roy, Newton Highlands, MA (US); Michael A. Ridge, Cambridge, MA (US); Scott E. Lennox, Arlington, MA (US); Rami Mangoubi, Newton, MA (US); Murali V. Chaparala, Newton, MA (US)

(21) Appl. No.: 16/114,838

(22) Filed: Aug. 28, 2018

Related U.S. Application Data
(60) Provisional application No. 62/551,577, filed on Aug. 29, 2017.

(51) Int. Cl. G16H 50/30; G06N 3/08; G16H 15/00; G16H 10/60

(52) U.S. Cl. CPC G16H 50/30; G16H 10/60; G16H 15/00; G06N 3/08

(57) ABSTRACT

A distributed analytics system for identification and determination of disease and/or injuries is implemented on mobile computing devices carried by the users and a distributed computer network communicating with the mobile computing devices.

### RELATED APPLICATIONS

This application claims the benefit under 35 USC 119(e) of U.S. Provisional Application No. 62/551,577, filed on Aug. 29, 2017, which is incorporated herein by reference in its entirety.

### BACKGROUND OF THE INVENTION

Use of handheld mobile computing devices that can access networks including the internet, typically in the form of cellular smartphones, is reaching the point where just about every person now has such a device.

These mobile computing devices, or user devices, are often equipped with multiple built-in sensors and/or subsystems that include sensors. The sensors generate signals, also known as raw sensor data, in response to detecting various phenomena that each sensor is designed to detect. The subsystems generally include one or more sensors and various components that provide power, enable exchange of control and data messages, and provide interfaces to an operating system of the user devices. These sensors include gyroscopes, accelerometers, barometers, and temperature sensors, in examples. The subsystems include global positioning system (GPS) and accelerometer-based subsystems, in examples.

Many of the mobile user devices further include near field communication (NFC) transceivers and Bluetooth transceivers that enable short-range connections with other user devices having the same capabilities.

Some of the subsystems of the user devices might be used to control medical devices worn or otherwise used by the individuals. The subsystems can control the medical devices when onboard control devices and local software of the user devices are connected to the medical devices. These connections often use communications networks and can be wired/tethered or wireless/untethered in nature. The communications networks typically use Universal Serial Bus (USB), WiFi, or other network protocols. Examples of medical devices that might be under control of the subsystems include pacemakers, insulin pumps, oxygen flow/concentration units, and possibly actuators and motors of wheel chairs, in examples.

The user devices also interface with sensors that are external to the user devices. The external sensors are generally worn on the person of the individuals. These external sensors typically have high sensitivity and specific usage. Examples of external sensors include fingertip oximeter sensors for measuring pulse, blood pressure cuffs for measuring blood pressure, and wristband/armband-based activity tracking devices such as “fitbit.” Fitbit is a registered trademark of Fitbit, Inc.

### SUMMARY OF THE INVENTION

Similar to how GPS-enabled mobile user devices provide real time traffic information, their many, still largely untapped, features can also provide information about their owners' physical and environmental conditions. The physical conditions of the individuals include medical conditions/symptoms such as shortness of breath, traumatic brain injury, epilepsy, diabetes, partial paralysis, hearing issues, sight limitations, medication usage, arrhythmia and other heart conditions, autism conditions, mental impairment, schizophrenia, and dementia. The environmental conditions include motion of the individuals tracked over time. This information can serve to diagnose their medical health, such as to provide early warning of disease and/or injuries.

As transceiver nodes of a health information network, the mobile user devices could provide information about their owners' health, and also receive messages about potential and existing public health concerns to inform the owners. A network of connected mobile user devices can thus become a vital public warning system for the health of entire populations, and each mobile user device can become an invaluable health diagnostic tool.

The proposed distributed analytics system can provide rapid and early identification of infectious diseases and injuries suffered by individuals. For this purpose, the system utilizes sensors on individual mobile user devices to unobtrusively (possibly without using personal identifiable information) detect and monitor conditions relevant for individual and public health, including classifying and identifying early signs of infections and injuries. In one example, the system can operate as an early public health warning system.

The mobile user devices of the system could utilize any available communications networks. Such networks include formal and informal networks such as peer-to-peer communications networks or ad-hoc networks. The ad-hoc networks can leverage the existing hardware (primarily Bluetooth and Wi-Fi transceivers) in commercially available mobile user devices to create peer-to-peer networks without relying on cellular carrier networks, wireless access points, or traditional network infrastructure.

The mobile user devices in the system detect and select the communications networks based on quality of service (QoS) calculations of one or more of available network protocols. QoS calculations include range calculations, network speed, power usage and connectivity required.

Each mobile user device will use (based on its model) some or all available communications protocols to form the communications networks and to communicate with other components of the system. Example communications protocols include: UWB, IEEE 801.11/WiFi, IEEE 802.16/WiMax, IEEE 802.15.4/ZigBee, Bluetooth, NFC/RFID, Land Mobile Radio, and cellular 2G/3G/4G/5G.

In a large deployment scenario (>10 million devices, in one example), the system can accurately perform predictive analysis and correlations of the physical and environmental conditions of individuals in real time, and can track trajectories of various conditions even in case of large movement/reorganizations within the population of the mobile user devices. The system is also cost effective at large scale, requiring only a software download on the user devices.

The subsystems and sensors of the mobile user devices that may participate in gathering data include: finger-print scanner, heart rate monitor, red/green/blue (RGB) ambient light measuring camera, temperature sensors (environmental and body, if the latter is present on the mobile user device), barometer, NFC, gyroscope, accelerometer, Bluetooth transceiver, WiFi receiver, FM radio receiver, cellular transceiver, front and rear cameras, GPS chipset, magnetic sensor, light flux sensor, battery temperature sensor, microphone and touch sensor, in examples.

In general, the system includes mobile user devices connected to a distributed computer network for monitoring, classifying and identifying early signs of infections and injuries. The distributed computer network includes computing devices such as one or more servers/system controllers located in one or more remote networks.

The system controllers, such as servers or cloud systems, host global software that communicates with the local software on each of the user devices. The global software executes on/across one or more computer processing units (CPUs) of the system controllers. The global software includes software modules/components such as a command and control module, and an artificial intelligence (AI) module.

In addition, the system can monitor an origin and spread/propagation of infectious diseases in real time.

In general, according to one aspect, the system is scalable to possibly millions of mobile user devices. The system is based on a parallel learning architecture that will share information learned from small sets of the mobile user devices with the rest of the mobile user devices.

At the distributed computer networks, the global software can be hosted with redundancy, high availability and collaborative control. In addition to the hosts being servers and/or system controllers, the hosts can be cloud-based server systems such as Amazon Web Services (AWS) and similar cloud data storage and compute platforms, mobile ruggedized field deployable/high performance computing platforms, and data centers, in examples.

The system preferably uses artificial intelligence (AI) based software applications on each mobile user device. These AI applications perform unsupervised sensor signal processing, new learning, condition detection and classification after deployment on training.

The system generally requires minimal training datasets. Large scale signal simulations (using high performance computing, or HPC) to enhance training datasets are used to train software deployed on each mobile user device. Identification and classification of non-degenerate basis vectors that form the feature vector set is now accurate, as large scale sensor simulations enable sampling of the feature vector space with high coverage. One or more of the feature vectors can now be used to detect one or more multiple conditions.

Preferably, the system optimizes the use of resources regarding computing capacities and bandwidth on the mobile user devices. In one example, the system preferably uses no more than 5% of total available power of each mobile user device. The system can leverage rapid application and deployment.

The AI application on each mobile user device produces a local neural network from a neural network model. The neural network model can be updated by the global software of the distributed computer network, or in response to new information received at each of the user devices. The global software includes a command and control module that updates the neural network model on each user device.

At the level of the user devices, the AI applications can also update the neural network model. The neural network model can be updated in response to new learning, upon new feature vector detection and classification for a given condition, upon receiving new sensor data from the sensors and/or subsystems, and/or upon new sensor sensitivity and calibration on the user device. The system preferably uses minimal data transfers from the mobile user devices by implementing a new and novel differential update algorithm with real time QoS (Quality of Service).

The system's local and global application and databases, which can run on IOS/Android locally and on Windows/Linux operating systems globally, preferably employ a cross-platform integrated development environment. The system can also be deployed and used in passive mode where, in the absence of cellular networks, peer-to-peer ad hoc network connections between the local mobile user devices are possible.

In general, according to one aspect, the invention features a distributed analytics system for identification, determination and early warning of disease and/or injuries. The system includes mobile computing devices carried by users that monitor the users via sensors, and a distributed computer network communicating with the mobile computing devices.

Preferably, the mobile computing devices include computer processing units executing artificial intelligence (AI) applications and sensors that provide sensor data to the AI applications. The AI applications executing on the computer processing units include neural networks.

The AI applications can also create training datasets that include the sensor data and send the training datasets for processing by a principal component analysis system of the distributed computer network that determines principal components. Typically, the principal components include feature vectors, basis vectors, and eigenvectors that are used by a training system to create model training data for a neural network model for the neural networks executing on the computer processing units of the mobile computing devices.

In one implementation, new sensor data from the sensors are used to train and/or update the neural networks of the user devices. Additionally, the mobile computing devices can locally update the neural networks when communications between the distributed computer network and the devices are lost.

Also in the system, data connections between the mobile computing devices and the distributed computer network might be created dynamically by the mobile computing devices including peer-to-peer ad hoc connections to other mobile computing devices.

The distributed computer network includes a differential update system, a diagnosis system, and a training system. In one example, the diagnosis system creates medical condition reports for individuals carrying the mobile computing devices.

In general, according to another aspect, the invention features a method for identification, determination and early warning of disease and/or injuries. The method comprises monitoring users with mobile computing devices carried by the users, and a distributed computer network communicating with the mobile computing devices for facilitating the identification, determination, and early warning of disease and/or injuries in the users.

In general, according to another aspect, the invention features a distributed analytics system. It comprises a command and control module that deploys neural network models to user devices and updates the neural network models in response to sensor data sent from sensors of the mobile user devices and one or more networks that provide communications between the command and control module and the mobile user devices.

The above and other features of the invention, including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a block diagram of a distributed analytics system for determining physical and environmental conditions of individuals carrying mobile user devices, according to the invention, where sensor data from sensors of the mobile user devices and other information are shown;

FIG. 2A is a block diagram showing more detail for an exemplary user device in FIG. 1, where a central processing unit (CPU), an operating system, memory, and local software of the user device are shown;

FIG. 2B is a block diagram showing more detail for a system controller in FIG. 1, where a CPU, an operating system, memory, and global software of the system controller are shown;

FIG. 3A is a sequence diagram that visually depicts an exemplary use case of the system in FIG. 1, as a public health warning system and emergency information provider;

FIG. 3B is a sequence diagram that visually depicts operation of a differential update system of the system in FIG. 1, by way of an example;

FIG. 4 is a schematic diagram that shows a recognition system with machine learning executing on a central processing unit, or graphic processing unit, or special purpose process of the mobile user device, where the diagram also shows interaction between the recognition system and the differential update system;

FIGS. 5A and 5B are normalized amplitude plots that illustrate how artificial intelligence (AI) applications executing on the mobile user devices extract feature vectors from the raw sensor data, where FIG. 5A shows how the mobile AI applications might sample the raw sensor data in the time domain to extract feature vectors with sample size=n, window size=W, and FIG. 5B shows the extracted features after smoothing using median filtering;

FIG. 6 shows more detail for an exemplary principal component analysis (PCA) system of the system of FIG. 1 that analyzes the sensor data sent from the mobile user devices;

FIG. 7 shows more detail for a trained neural network of the AI application on the exemplary mobile user device in FIG. 4;

FIG. 8 is a plot of analysis of sensor data over time that is used to determine the motion state of the user based on the mobile user device's sensors;

FIG. 9 is a schematic diagram showing differential updates of sparse data from each mobile user device, and software updates from a controller of the system to the mobile computing devices;

FIG. 10 shows consumption of power (residual in power level with a starting level of 60% power) of the mobile user device after 40 minutes of sensor use, followed by 50 minutes of sensors plus WiFi, and followed by 50 minutes of sensors, WiFi and GPS; and

FIG. 11 and FIG. 12 are block diagrams that illustrate different operational models for how the AI application executing on each mobile user device combines (“fuses”) the sensor data from the sensors, where: FIG. 11 shows that the AI application first fuses the sensor data, and then formats the fused sensor data for subsequent analysis by the system controller; and

FIG. 12 shows how the sensor data from the sensors are individually processed/formatted and then fused prior to transmitting the sensor data to the system controller for analysis.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

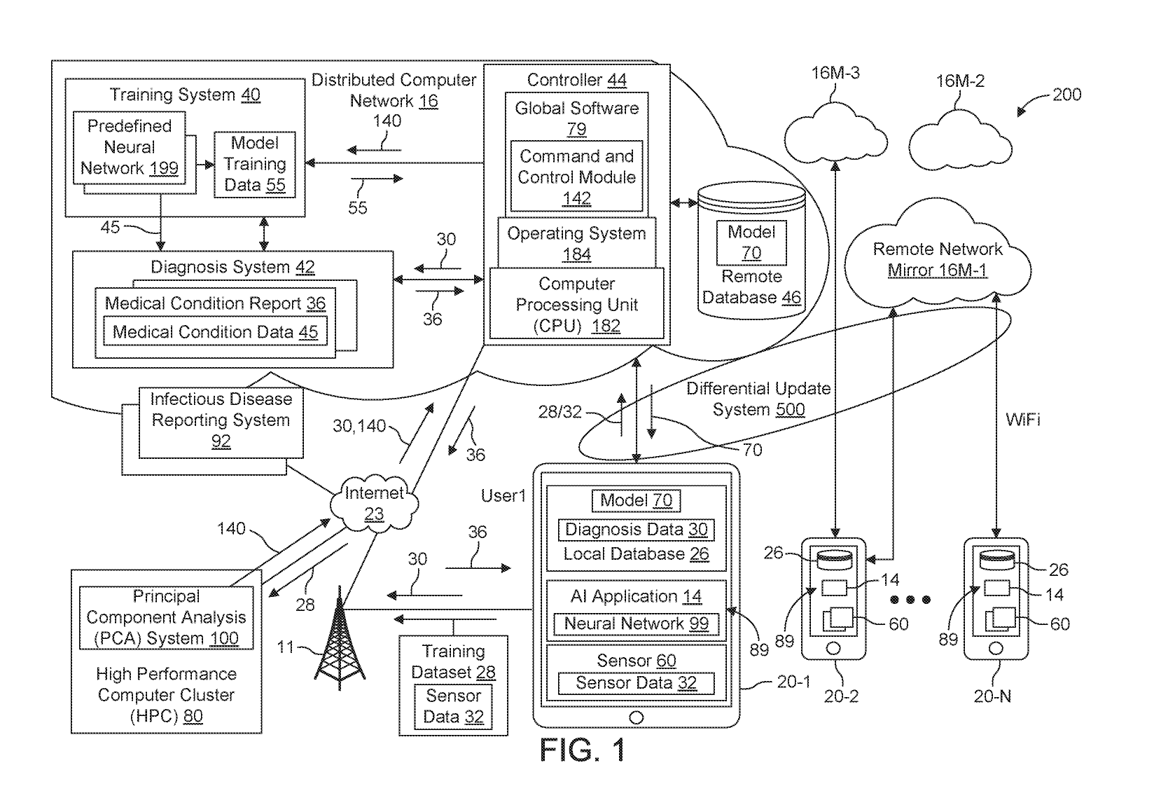

FIG. 1 shows an exemplary distributed analytics system 200, which has been constructed according to the principles of the present invention.

In general, data flows from various components of the system are shown, from initial data acquisition by mobile user devices to determination of conditions of individuals.

The system 200 has various components. These components include user devices 20-1 through 20-N, a distributed computer network 16 and remote network mirrors 16M, a high performance computer cluster 80, and a differential update system 500.

Three remote network mirrors 16M-1, 16M2, and 16M3 are shown. The remote network mirrors 16M, as their name suggests, are parallel communications networks to the distributed computer network 16. The remote network mirrors 16M provide alternate communications paths to the user devices 20 in the event that the distributed computer network 16 fails, is overutilized, or when the distributed computer network does not have a direct connection to the various mobile communications devices 20. Typically, the remote network mirrors 16M-1, 16M2, and 16M3 and the distributed computer network 16 are interconnected via a high speed communications backbone.

The distributed computer network 16 has various components and connects to both the internet 23 and the differential update system 500. These components include a diagnosis system 42, a training system 40, a controller 44, and a remote database 46. The controller operates as a system controller of the distributed computer network 16 and also provides the connection to the internet 23.

The controller 44 includes one or more computer processing units (CPUs) 182, an operating system 184, and global software 79. In the illustrated example, a command and control module 142 of the global software 79 is shown. The controller 44, via its command and control module 142, creates a neural network model 70 for deployment on the user devices 20.

The user devices 20 each include a local database 26, an AI application 14, and sensors 60. Detail for exemplary user device 20-1 is shown. Specifically, the local database 26 includes diagnosis data 30 and the neural network model 70 sent from the command and control module 142. Also shown is a neural network 99 that the AI application 14 creates from the model 70.

The sensors 60 acquire information from the physical environment for which the sensors were designed, and store this information as the sensor data 32.

In the illustrated example, the user devices 20 communicate over radio frequency links including both WiFi and cellular networks. Preferably the devices will utilize available networks such as those based on UWB, IEEE 801.11/WiFi, IEEE 802.16/WiMax, IEEE 802.15.4/ZigBee, Bluetooth, NFC/RFID, Land Mobile Radio, and cellular 2G/3G/4G/5G to establish adequate data transfer rates while minimizing power consumption.

User device 20-1, for example, has a cellular network interface that connects to a tower 11 of a cellular network. The tower 11, in turn, connects to the internet 23. The user devices 20 also have WiFi interfaces that connect the user devices to the differential update system 500.

The diagnosis system 42 can create medical condition reports 36 for individuals carrying the user devices 20, and to public health officials and to medical companies performing medical device and medication trials and patient monitoring. The diagnosis system 42 determines medical condition data 45 of the individuals in response to analyzing information sent from the user devices 20. The diagnosis system 42 includes the medical condition data 45 in anonymized medical condition reports 36 for the individuals, and can also store the medical condition reports 36 to the remote database 46 via the controller 44.

The HPC 80 includes a principal component analysis system (PCA) 100. The PCA receives training datasets 28 sent by multiple user devices 20. Using content within the training datasets 28, the PCA can simulate content of training datasets 28 from future user devices 20. The PCA determines principal components of the content (i.e. sensor data) for subsequent use by the training system 40. The principal components represent the sensor data 32.

The system 200 also communicates with infectious disease reporting systems 92 over the internet 23. One exemplary infectious disease reporting system is that of the U.S. Center for Disease Control (CDC) in Atlanta, Ga.

The diagnosis system 42 also generates a medical condition report 36 from the diagnosis data 30 for each user and sends the personal medical condition report 36 to the user device 20-1 of user1, for example.

The training system 40 includes one or more predefined neural networks 199 and generates model training data 55. Each of the predefined neural networks 199 typically correspond to different neural network types. The command and control module 142 generates an initial neural network model 70 for deployment on the user devices 20, based upon the one or more predefined neural networks 199 and the model training data 55.

The system 200 generally operates as follows. An individual such as user 1 is carrying user device 20-1, and sensors 60 of user device 20-1 acquire sensor data 32 that is in the form of time-domain signals. Initially, the user devices 20 typically do not include the neural network model 70, and thus also do not include the neural network 99 that the AI application 14 generates from the model 70.

The AI application 14 creates a training dataset 28 that includes the sensor data 32, and sends the training dataset 28 for processing by the PCA system 100 of the HPC 80. The AI application 14 might also send the diagnosis data 30 of the user for analysis by the diagnosis system 42. The AI application 14 also sends raw or pre-processed sensor data 32 to the command and control module 142 of the controller 44.

At the HPC 80, the PCA system 100 determines principal components of the content of the training dataset 28.

The principal components include feature vectors, basis vectors, and eigenvectors, in examples. The principal components also include a knowledge base with rules and axioms in conjunctive normal form from first-order logic rules. The basis set of these rules when satisfied by the neural network (the knowledge and inference system is neural network type agnostic) is also part of the feature vector and basis vector set. For example, a basis vector (eigenvectors) for the detection of autism would be a set of correlation times and frequencies that would repeat in a certain pattern. When a signal (that has this signature) is fed into the trained neural network 199 at the training system 40, the training system 40 creates the model training data 55 as output that would satisfy the basis set of rules and axioms in the knowledge base for the given condition. Simulated signals could also be created using the basis vectors that satisfy the basis in the knowledge base to augment/test the training set.

In one example, when the signals of the sensor data 32 are from accelerometers and GPS chipsets as the sensors 60, the principal components are also known as “conduct signals” 140. The conduct signals 140 and the diagnosis data 30 are then sent to the distributed computer network 16 for further processing.

The training system 40 constructs its model training data 55 based upon a predefined neural network 199 and in response to the conduct signals 140 calculated and sent from the PCA system 100.

Then, at the controller 44, the command and control module 142 generates the initial neural network model 70 for deployment on the user devices 20 from the model training data 55. The command and control module 142 then sends the model 70 for deployment on the user devices 20 via the differential update system 500. The controller 44 also saves the model to the remote database 46.

Over time, new sensor data 32 from the sensors 60 are received at the user devices 20. The new sensor data 32 is then used to train and/or update the neural network model 70 on each of the user devices 20. For this purpose, either the command and control module 142 or the user devices 20 themselves can update the model 70 in response to new sensor data/updates to the sensor data 20.

In a preferred embodiment, the command and control module 142 provides an updated model 70 to the user devices. Here, the user devices 20 send updates to the sensor data over the differential update system 500 to the command and control module 142, and the command and control module 142 provides an updated model 70 in response. The command and control module 142 then deploys the updated model 70 to the user devices over the differential update system 500. Alternatively, in another embodiment, once the command and control module 142 has deployed an initial model 70 on the user device 20, the AI application 14 on the user devices 20 can update the model 70 locally.

When the command and control module 142 updates the model 70, the user devices 20 determine a preferred communications path to the controller 44 when sending the new or updated sensor data 32. The user devices 20 determine a preferred communications path based on the quality of service (QoS) of the available communications links between the user device 20 and the controller 44 and the power available at the user device 20. Additionally, the sensor data 32 may be preprocessed at the user devices 20 before sending to the command and control module 142.

When the user devices send the sensor data 32 to the command and control module 142, all updates are differential. The user devices 20 send only new data, learning or raw data 32 that was not sent earlier. The command and control module 142 then updates the model 70 based upon the sensor data 32 and/or learning sent over the differential update system 500 by the user devices.

When a cellular network is available with suitable QoS, in one example, the local AI application 14 executing on each of the user devices switches to an active scanning mode and the global software 79 takes over communication to each mobile user device. Peer-to-peer ad hoc connections to other user devices 20 are now disabled locally.

When the AI application 14 is in its active scanning mode, the AI application 14 operates as follows. The AI application 14 obtains or uses the sensor data 32 from the sensors 60 and new learning performed locally by the AI application 14 to determine the best way to transmit the sensor data 32 and learning to the command and control module 142. The local software 89 then determines which network connections to the command and control software 142 have the best quality of service. Upon selecting a network connection to the command and control software 142, the AI application 14 typically hands over control of the communications session to the command and control software 142.

At the controller 44, the command and control software 142 then manages the communications session between the command and control software 142 and the AI application. In this way, when there are breaks in the communications session, drops in power at the user devices 20, or quality of service issues like low bandwidth, the local software 89 does not need input from the command and control software 142 to continue with tasks and new learning. Thus, learning is transferred to the command and control software 142 with the local software doing it locally if power is not an issue. Otherwise, the new active scanned data/or saved sensor data 32 is sent via a differential update to the controller 44, and new learning is performed by the AI module 144 and pushed back to the local software 89 on the user devices 20.

Connections between the global software 79 of the distributed computer network 16 and the user devices 20 are created dynamically by the local software 89 on the user devices 20. The global software 79 knows and tracks QoS, protocols and related information of the distributed computer networks 16. The global software 79 also forecasts and decides which networks the local software 89 on the user devices 20 may use.

The communications between the global software 79 executing on the CPU 182 of the controller 44 and the local software 89 executing on the user devices 20 are as follows. The communications include software updates and new learning sent from the command and control module 142 to the local software 89, differential sensor data 32 updates sent from the local software 89 to the global software 79, and peer-to-peer ad hoc communications between the user devices if required by the user devices 20 or the local software groups. The communications protocols used during the communications are selected dynamically by the local software 89 depending on availability, bandwidth requirements and range as well as power considerations.

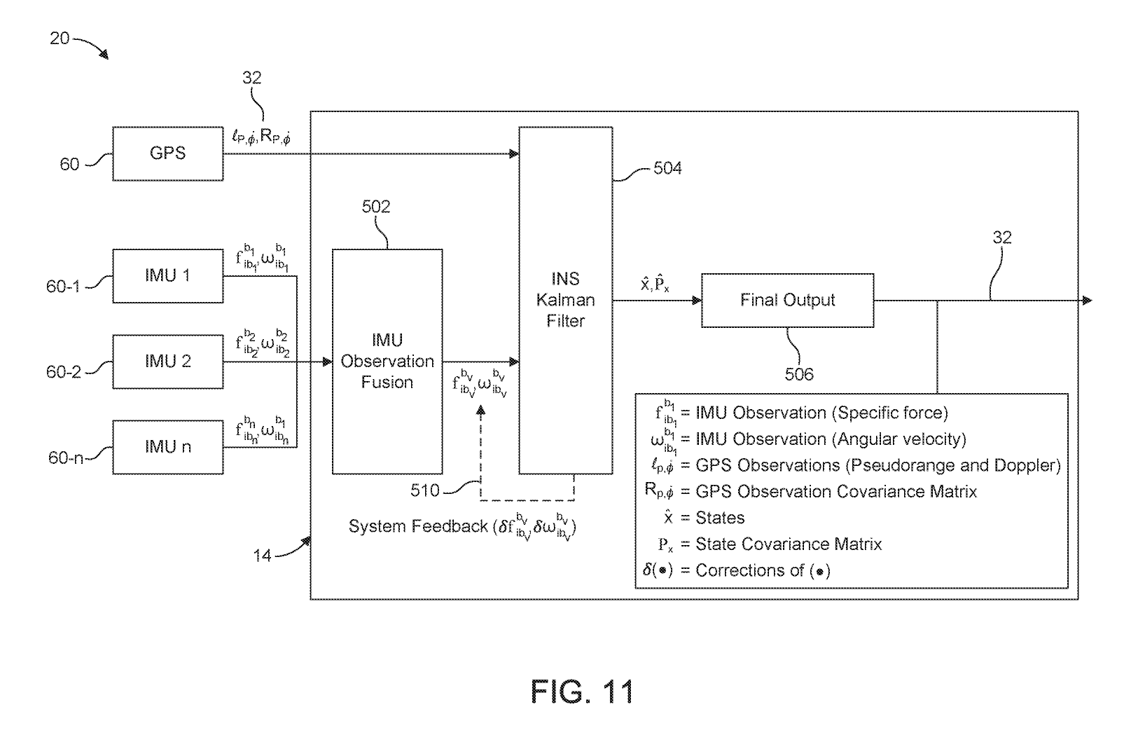

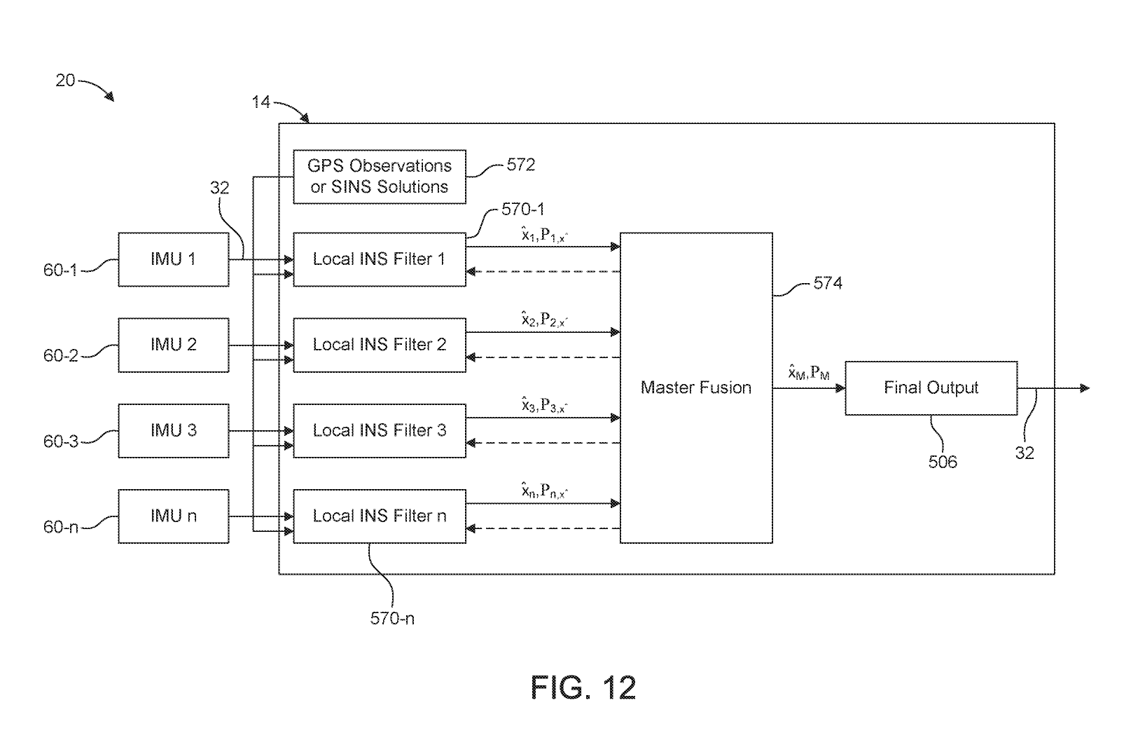

When the AI application 14 on the user devices 20 updates the model 70 locally, the AI application 14 typically combines/“fuses” the sensor data 32 from multiple sensors 60. The AI application 14 then generates the updated model 70 from the fused sensor data. More information regarding different fusion models of the AI application 14 is in the description accompanying FIGS. 11 and 12, included hereinbelow.

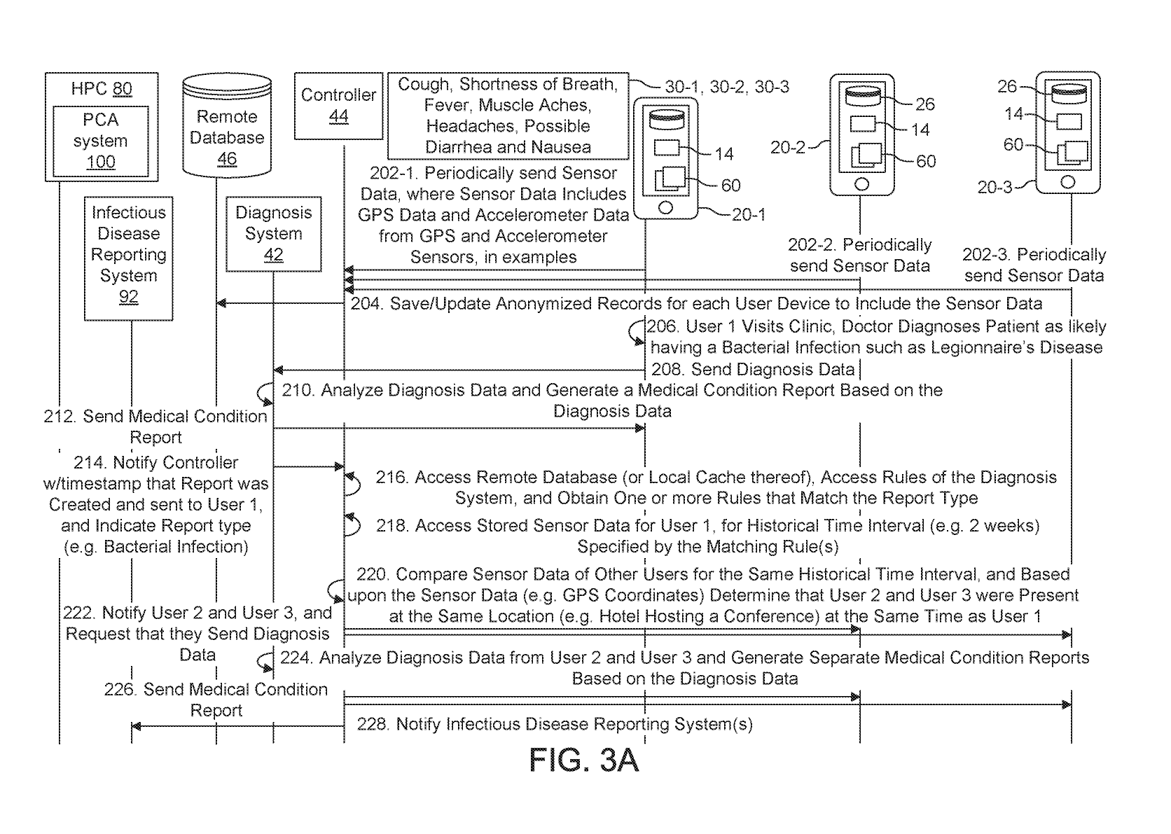

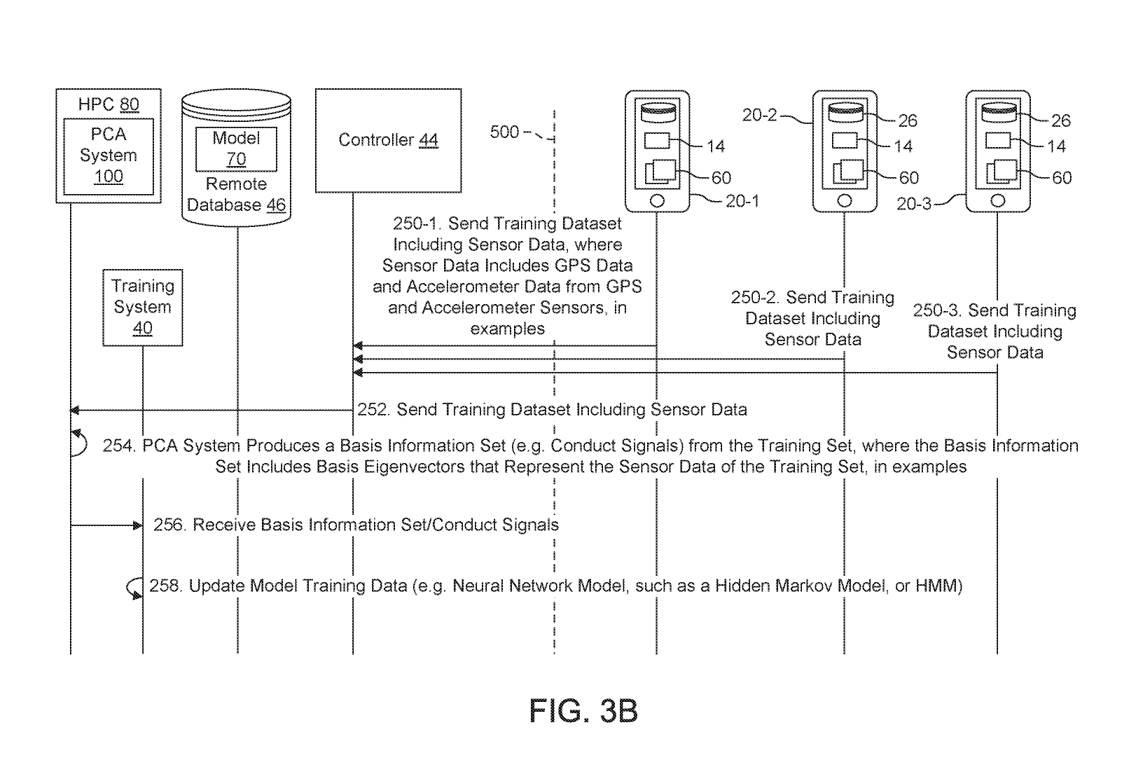

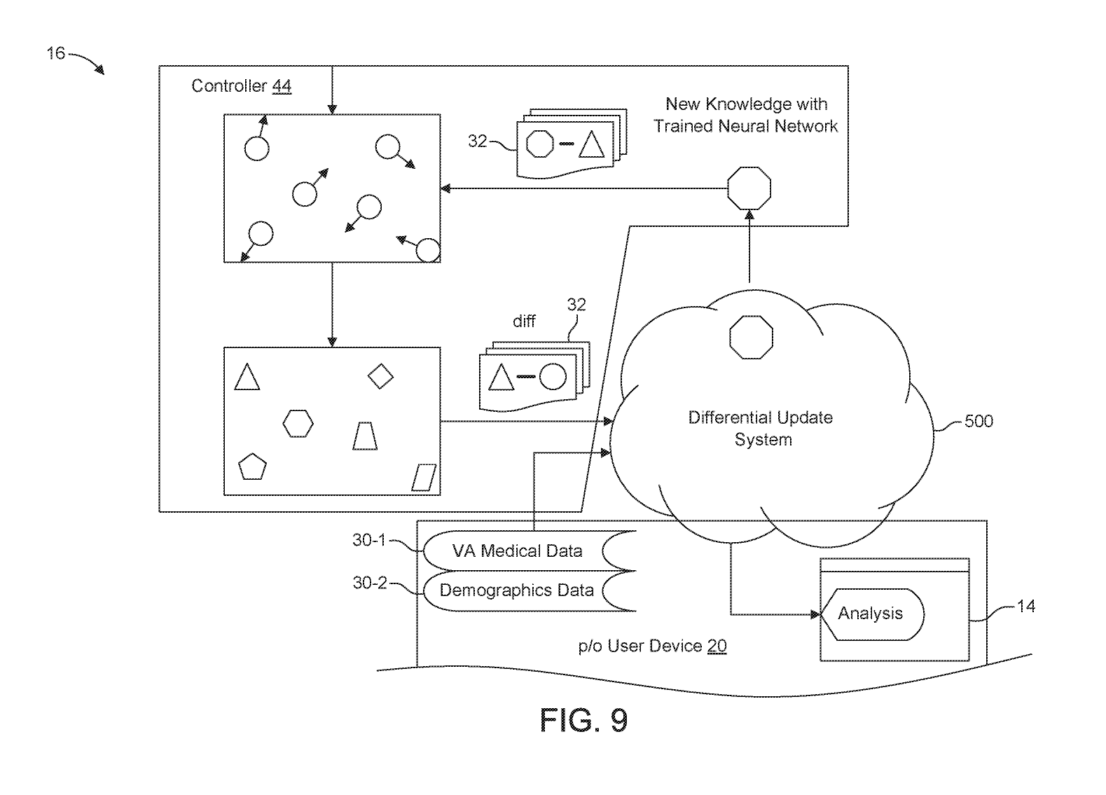

The user devices 200 can then update their local models 70 from the newly generated model 70 at the distributed computer network 16, also sent via the differential update system 500. The differential update system 500 is described in more detail in the descriptions that accompany FIG. 3B and FIG. 9, included hereinbelow.

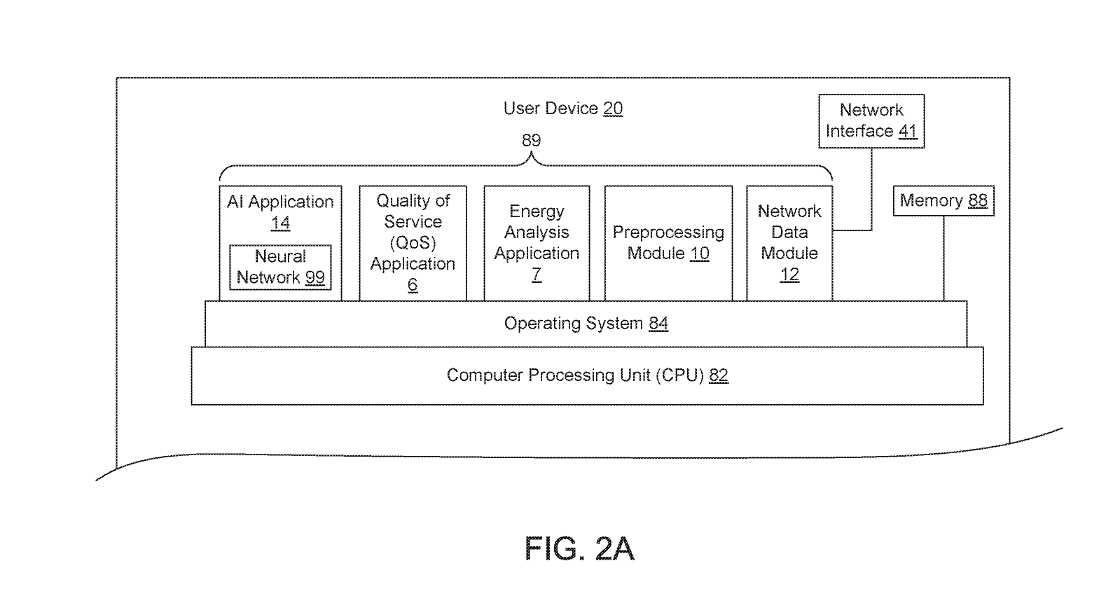

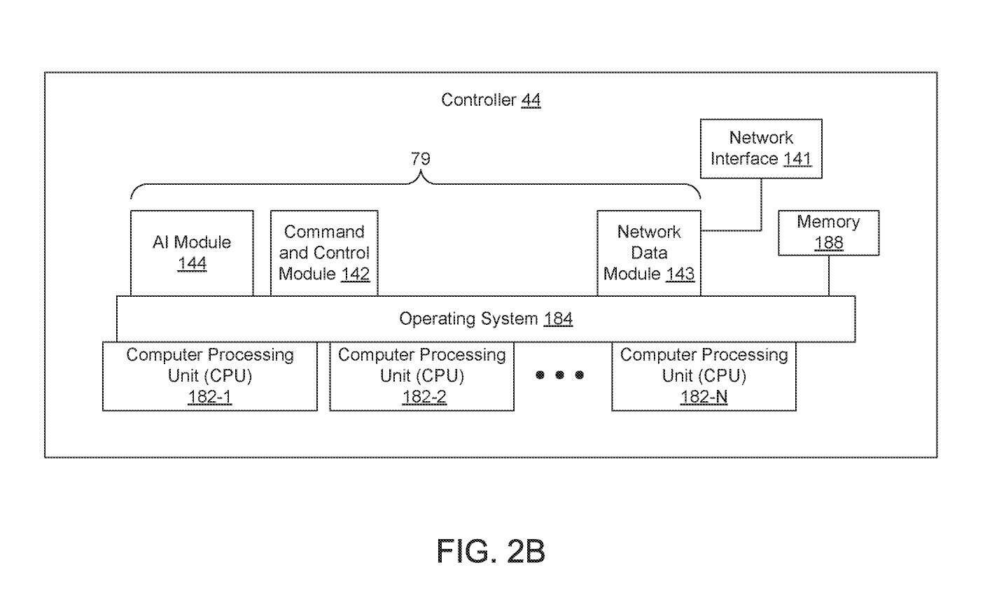

FIG. 2A shows more detail for an exemplary user device 20. Specifically, the figure shows detail for local software 89 and components that enable execution of the local software 89 on the user device 20.

In the illustrated example, the user device 20 includes an operating system 84, the local software/applications 89, memory 88, a network interface 41, and one or more computer processing units (CPUs) 82. The operating system might be IOS (typically on devices manufactured by Apple Corporation) or an Android operating systems (typically on non-Apple devices). Android is a trademark of Google, Inc. The CPUs 82 may include multiple independent cores, may support multithreading, and might include graphics processing units or special purpose processing units as co-processors, in examples.

The local software 89 includes the AI application 14, a quality of service (QoS) application, an energy analysis application 7, a preprocessing module 10, and a network data module 12.

The operating system 84 loads the local software 89 into the memory 88 and schedules the local software 89 for execution by the CPU 82. The preprocessing module 10 captures the sensor data 32 from the sensors 60 and performs various preprocessing operations on the sensor data 32 before providing the sensor data to the AI application 14.

The user devices can access networks via the network data module 12 and the network interface 41. The network data module 12 receives information from one or more network connections via the network interface 41, and sends information to the network interface 41 for transmission over the one or more network connections. These network connections can be associated with ad-hoc networks formed between the user devices 20, connections to the distributed computer networks 16, and connections of differential update system 500, in examples. In one example, the user devices 20 download the local software 89 from the controller 44 via the internet 23.

FIG. 2B shows more detail for the controller 44. Specifically, the figure shows detail for global software 79 and components that enable execution of the global software 79 on the controller 44.

In the illustrated example, the controller 44 includes an operating system 184, the global software 79, memory 188, a network interface 141, and one or more computer processing units (CPUs) 182-1 . . . 182-N. The operating system might be Windows or Linux-based, in examples. The CPUs 182 may include multiple independent cores, may support multithreading, and might be graphics processing units or special purpose processing units, in examples.

The global software 79 includes an AI module 144, a command and control module 142 and a network data module 143.

The operating system 184 loads the global software 79 into the memory 188 and schedules the global software 79 for execution by the CPU(s) 182.

The controller 44 can access networks via the network data module 143 and the network interface 141. The network data module 143 receives information from one or more network connections via the network interface 141, and sends information to the network interface 141 for transmission over the one or more network connections.

FIG. 3A is a potential use case for the system 200 as an early health warning system. Here, mobile devices 20A, 20B, and 20C are carried by persons A, B, and C. The mobile devices 20A, 20B, and 20C are respectively reporting diagnostics data 30-1, 30-2, and 30-3 of individuals user1, user2, and user3, respectively.

As an example, users1, 2, and 3 had previously attended a conference at the same hotel a week earlier. User1 visits a doctor, complaining of symptoms such as a cough, shortness of breath, fever, muscle aches, headaches, possible diarrhea and nausea.

In steps 202-1 through 202-3, the user devices 20-1 through 20-3 periodically send sensor data 32 to the controller 44. Here, the sensor data 32 includes GPS data and accelerometer data from GPS and accelerometer sensors, in examples. According to step 204, the controller 44 saves/updates anonymized records for each user device/user to include the sensor data 20 at the remote database 46.

At step 206, user1 visits a clinic. A doctor at the clinic diagnoses user1 as likely having a bacterial infection such as Legionnaire's disease. Using the AI application 14, the user1 or the doctor enters and stores these symptoms as diagnosis data 30 on user device 20-1 and sends the diagnosis data to the diagnosis system 42 in step 208. In step 210, the diagnosis system 42 analyzes the diagnosis data 30, and generates a medical condition report 36 including medical condition data 35 based on the diagnosis data 30. In step 212, the diagnosis system 42 sends the medical condition report 36 to user1/user device 20-1.

According to step 214, the diagnosis system 42 notifies the controller 44 w/timestamp that the report 36 was created and sent to user 1, and indicates a report type (e.g. bacterial infection). Then, in step 216, the controller 44 accesses the remote database 46 (or local cache thereof), accesses rules of the diagnosis system 42, and obtains one or more rules that match the report type.

In step 220, the controller 44 compares the sensor data 32 of other users for the same historical time interval. Based upon the sensor data 32 (e.g. GPS coordinates), the controller 44 determines that both user2 and user3 were present at the same location (e.g. hotel hosting the conference) at the same time as user1.

In response, in step 222, the controller 44 notifies user2 and user3, and requests that they send their diagnosis data 30-2 and 30-3, respectively. In step 224, the controller 44 analyzes the diagnosis data 30-2 and 30-3 from user2 and user3, and generates separate medical condition reports 36 based on the diagnosis data 30-2/30-3. The controller in step 226 sends the medical condition reports 36 to user2 and user3, and notifies the infectious disease reporting system(s) 92.

FIG. 3B is a sequence diagram that visually depicts operation of the differential update system 500, by way of another example.

Here, an initial neural network model 70 has already been generated by the command and control module 142 and deployed on the user devices 20. The user devices 20 are in “active scanning mode,” where the user devices 20 are actively receiving sensor data 32 from their sensors 60. The user devices 20 preprocess the sensor data 32, and send the updated sensor data 32 in training datasets 28 over the differential update system 500 to the controller 44 for further processing.

In steps 250-1 through 250-3, the user devices 20-1 through 20-3 send sensor data 32 to the controller 44. Here, the sensor data 32 includes GPS data and accelerometer data from GPS and accelerometer sensors, in examples. The user devices 20 also send training datasets 28 to the controller, the training datasets 28 including the sensor data 32 from each of the user devices 20. According to step 252, the controller 44 sends the training datasets 28 including the sensor data 32 to the HPC 80 for analysis.

According to step 254, the PCA system 100 produces a basis information set (conduct signals 140) from the training datasets 28, where the basis information set includes basis eigenvectors that represent the sensor data 32 of the training dataset 28.

The principal component analysis performed by the PCA system 100 depends on the training and the knowledge base (i.e. the sensor data 32). Initially, at system startup, there is no knowledge base/information to train, and thus the principal components/conduct signals 140 include only some basic information of the vectors and axioms. As the training proceeds, the conduct signals 140 are built up until there is no more improvement in the trained model. As a result, a valid basis set is created (PCA decomposition is complete).

In step 256, the training system 40 receives the basis information set/conduct signals 140. In step 258, the training system 40 updates its model training data 55 (e.g. neural network model, such as a Hidden Markov Model, or HMM.)

The differential update system 500 typically operates in two different modes. These modes include a training/learning mode and a learned/deployed mode. More detail for these operational modes are included below:

1) Training learning Mode: A sampling of the sensor data 32 is performed by the preprocessing module 10 over a sampling window at each user device, and the sampled sensor data signal 32 is uploaded to the global software 79. For this purpose, the sampled sensor data 32 is typically included in a training dataset 28 that the user devices 20 send to the global software 79 at the controller 44.

Then, in another sampling window of the preprocessing module 10, more sensor data 32 from the sensors 60 are captured and sampled. Here, however, the preprocessing module 10 on the user device 20 does not need to send the entirety of the sampled sensor data 32 signals of the current sampling window. Instead, the preprocessing module 10 typically sends only information that is new or different, compared to the previous sampled sensor data signals/previous sampling window.

Different cross-correlation functions and transformations are used to determine the differences in the sampled sensor data signals of the current and previous sampling windows. The sampling is redone (resampled) so signals match. Then, only the cross-correlated difference signal is sent to the command and control module 142. At the command and control module 142, the difference signal is reconstructed and used.

At the user device 20, the new cross-correlated difference signal is saved for the next sampling window. Thus, storage constraints on the user device 20 are also considered in how the signal is saved—pure signals or the correlated difference signals. The sampling rate, number of sensors 60 and user device model type dictate how the training software on the user devices 20 processes and calculates the differential updates of the signals during the learning process.

2) Learned/deployed Mode: In this mode, the command and control module 142 provides a trained model 70 for execution/deployment on the user device 20 in an autonomous manner. Then, the user device receives and samples new signals/sensor data 32 from the sensors 60 based on output from the trained model 70. One of several alternatives are possible:

a) A condition is detected—send to the command and control module 142.

b) An unknown condition/state is reached—based on QoS, perform learning of the signal on the user device, or (preferred) send the signal to the command and control module 142 or do both. The command and control module 142 then checks the learning on the user device, if any, and proceeds to update the user devices with new learning after completing training cycles with simulated data to aid in accuracy.

c) Further sampling is necessary. If there are resources available (power, QoS, storage etc) on the user device, continue the sampling operations on the user device. Otherwise, the user device 20 sends the set of sampled signals to the command and control module 142 to perform further simulations.

Upon completing the simulations, the command and control module 142 reports back to the user devices 20. The command and control module 142 can then continue receiving and processing sampled sensor data 20 from the user devices, send a new trained model 70 to all user devices 20, or finish detection for the user device of the condition.

The use of the command and control module 142 in the differential update system 500 improves upon the computational complexity of sampling the sensor data 32 and ultimately processed to determine the physical and environmental conditions of the individuals. The command and control module 142 enables this process to be a polynomial time and space complexity problem, as compared to an NP complete problem in the absence of the command and control module 142. This is an innovative part of the system 200. The command and control module 142 performs software updates to the user devices 20 with the new trained models 70.

The QoS application 6 helps the local software 89 decide what to do locally and whether to let the command and control module 142 take over the majority of the work involved with processing the sensor data 32 from the sensors 60. If the connection between the local software 89 of a user device 20 and the global software 79 is down or otherwise poor in quality communication, the QoS application 6 of that user device determines other/better communications channels to the controller 44. Alternatively, if no other channels are available, the user device 20 can set up or join an ad-hoc communication network with other user devices 20 that in turn have a communications path to the global software 79/controller 44. In this way, the other user devices 20 in the ad-hoc network can report to the command and control module 142 on behalf of the user device 20 until direct communications between the user device 20 and the command and control module 142 is restored. All of these failover mechanisms are automated.

One example that illustrates operation of the system 200 is when the user devices 20 are carried by active military members, such as soldiers. In the example, the distributed computer network 16 and the HPC 80 are typically located at a military base within which the soldiers are normally stationed. Other soldiers are deployed away from the base in platoons, in a theatre of battle or in a training exercise. By comparing sensor data 32 from user devices 20 in the platoons against sensor data 32 from user devices carried by soldiers still stationed at the military base, the system 200 might determine whether the soldiers in the platoons are exposed to new biological or chemical agents and show a response, in one example.

Also in the military example, assume that the main communications channel between the user devices 20 in the platoons and the global software 79 at the military base is lost. In this case, the local software 89 on the user devices 20 in the platoons sets up an ad-hoc network between the user devices 20 at the military base and performs training locally to detect this. When the communications link between the user devices 20 in the platoons and the command and control module 142 is resumed, the user devices send a differential update of the sensor data 32 to the command and control module 142, and the new training is augmented by the command and control module 142 to produce a new neural network model 70. The command and control module 142 then deploys the new model 70 to the user devices of all the soldiers, both on base and in the platoons.

As a result, the new agent (biological or chemical) is now detected and the effects they produce are known. It may be case that in the next platoon deployment there is now an early warning system, detecting the effects much earlier.

The mobile user devices 20 in the system 200 preferably utilize all available sensors 60. Using the Samsung's Galaxy S series cellular phones as an example, the first model S included the following sensors: gyroscope, accelerometer, Bluetooth radio, WiFi radio, FM radio, cellphone radio, front and rear cameras, GPS, magnetic field, light flux, battery temperature, microphone, and touch/haptic. New sensors were then added to each successive Samsung Galaxy model SII, SIII, S4, and S5.

In more detail, the SII additionally included a gyroscope sensor. The SIII included all sensors as in the SII, and additionally included an NFC sensor and a barometer sensor. The S4 included all sensors as in the SIII, and also included an environmental temperature sensor, a relative humidity sensor, and an RGB ambient light sensor. Finally, the SS5 included all sensors as in the SIII, also included the RGB ambient light sensor, and additionally included a heartrate sensor and a fingerprint scanner sensor.

Similar sensors and the associated applications that interface with the sensors 60 are also available on iPhone devices. The system will be operational on cellular phones using Android (mostly non-Apple phone) and IOS (Apple phones) operating systems.

The system uses data from sensors, particularly accelerometers and GPS chipsets, on individual user devices or groups of user devices to unobtrusively (without using personal identifiable information) to detect and monitor conditions. The system serves as an early warning system.

The system in a large deployment scenario (>10 million user devices) accurately performs predictive analysis and correlations of conditions in real time and tracks trajectories of various conditions even in case of large movement/reorganizations within the population of the user devices.

The system 200 is cost effective at large scale, requiring only a software download and AI application 14 installation on the mobile user devices.

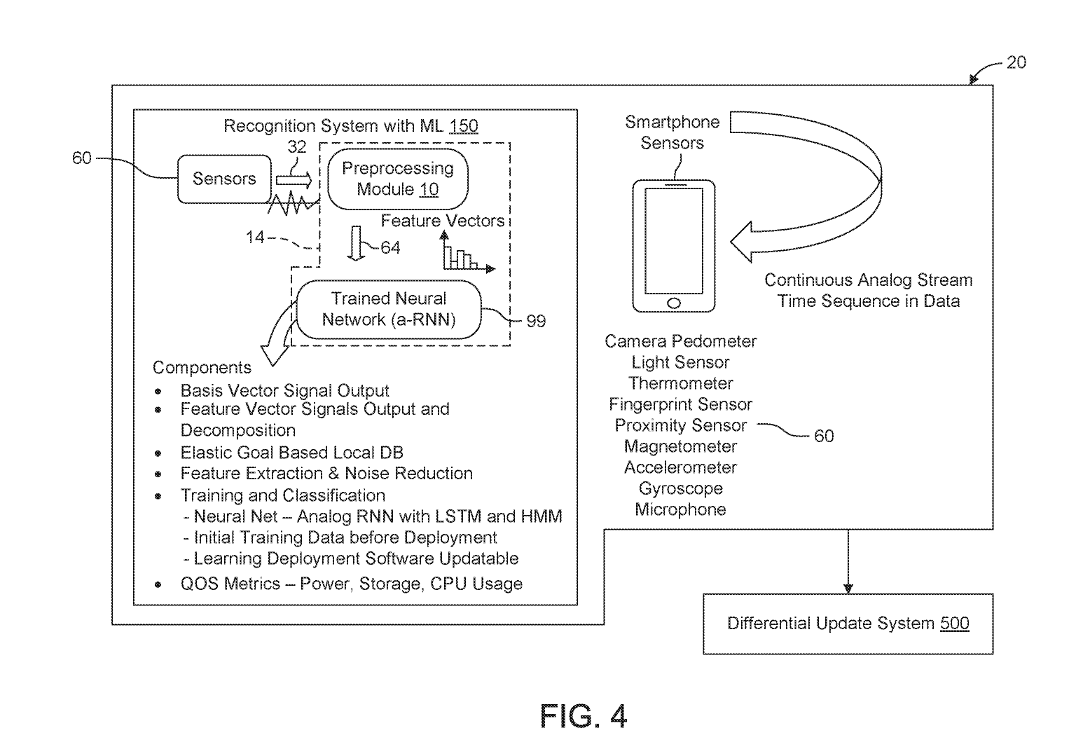

FIG. 4 shows the component blocks comprising the interaction of user devices 20 with the system detection/recognition software, along with the cloud and differential update system 500.

On the user device 20, a recognition system with machine learning (ML) 150 is shown. The recognition system with machine learning 150 is formed from the one or more device sensors 60 sending sensor data 32, a preprocessing module 10 of the AI application 14 producing feature vectors 64 from the sensor data 32, and a trained neural network 99 that is trained is response to receiving the feature vectors 64. Here, the trained neural network 99 is a hybrid analog recurrent neural network (a-RNN), according to one implementation.

The sensor data 32 for detection is typically preprocessed at the user devices 20, as shown in FIG. 4 in the box labeled “Preprocessing” 10. Alternatively, the preprocessing of the sensor data 32 can be performed by the global software 79 at the controller 44. Additionally, because both the command and control software 142 and the local software 89 as communications endpoints continuously monitor the communications link between the endpoints, the communications link provides a feedback loop between the endpoints.

The architectural component modules of the system 200 have the following components: 1. A global platform that has control software for training, deploying, monitoring, and updating handhelds and for collaborating; 2. Software run on each handheld 20; 3. A global database 46 accessed by the controller 44 that can be mirrored for locality and high availability; 4. Elastic goal based local database 26 on each handheld linked to the software (e.g. AI application 14) on each handheld 20; and 5. Handheld software such as the AI application 14 developed on global platform using C++ (debugged, tested and deployable on IOS or Android based handhelds using cross-platform deployment).

The system 200 uses supervised and unsupervised machine learning based algorithms at both the system controller 44 level as well as at the local handheld 20 software level.

The system 200 identifies the basis sets of the feature vectors accurately and trains the neural networks using experimental data and a much larger data set based on large-scale high-performance sensor signal simulations run on any large high-performance computational cluster.

A significant amount of this training is preferably performed at the command and control module 142 and/or AI module 144 of the global software 79. This is because the training generally creates a exponentially large set of simulated data. This training tests and augments the basis set of vectors in addition to learning the condition and the progression effects and related responses by the sensors 60.

The controller 44 is implemented as follows.

It is developed on a cross-platform software development environment or platform, for example. In one example, Microsoft Corporations “Microsoft Visual Studio Mobile App Development Platform” is used.

Sensor signal large scale simulations require the large high performance computational (HPC) cluster 80. In one example, an HPC cluster 80 has 512 compute cores of Intel Xeon E5-2620 v4 CPU's, 4 TB of global memory and 120 TB of storage that is cluster-wide (Ceph based file storage system). All are deployed on a high-speed trunked 10G network backplane. Compilers available include Intel, GNU 4.4, 4.8, 4.9 and 5.2.)

The controller 44 can be in the cloud or on any large computational cluster. Both the controller 44 and the user devices have a machine learning component. The command and control module 142 controls how much new machine learning is performed on the user devices 20, and whether the trained models are used directly on the user devices with no local new learning loops.

Training of the machine learning component of the global software 79 at the controller 44 (i.e. the AI module 144) and for a machine learning component of the local software 89 (i.e. the AI application 14) can be performed in the distributed computer network 16, on the HPC cluster 80, or upon any large memory server, in examples.

The machine learning component of the global software 79 is a series of trained neural networks with a knowledge base and an inference module. The location of the HPC 80 and its resources such as the PCA system 100 can be located anywhere in the system 200, as long as the HPC 80 is accessible by the command and control module 142 at all times.

During training there is flexibility in testing/training and obtaining experimental data from the user devices 20. In one example, the Amazon Mechanical Turk Application Programming Interface (API) might be used. In other examples, collaborations with governmental and private organizations, hospitals, (such as VA, Veterans Administration, hospitals) and health-care providers like community centers might also be leveraged.

During an automatic software refresh cycle on the user devices 20, new learning from some or all of the user devices 20 is used to refresh the local (learning) database 26 of every user device 20. Each user device 20 currently deployed is now capable of unsupervised learning. The (learnt) signals/sensor data 32 on each user device 20 form a unique sparse local dataset (local database).

The learning stage is parallelized across each user device. In one example, if the command and control module 142 decides to use the resources of the user devices 20 for preprocessing of the input signals and training, then learning is implemented across all of the user devices 20.

Updates to and from the distributed computer network 16 are done selectively and optimally to minimize streaming data traffic and to ensure scalability with respect to number of user devices deployed. The updates include new training models, new conditions, and new sampling of signals (raw or preprocessed), in examples. See the description of FIG. 9 that describes differential updating, included herein below, in one example.

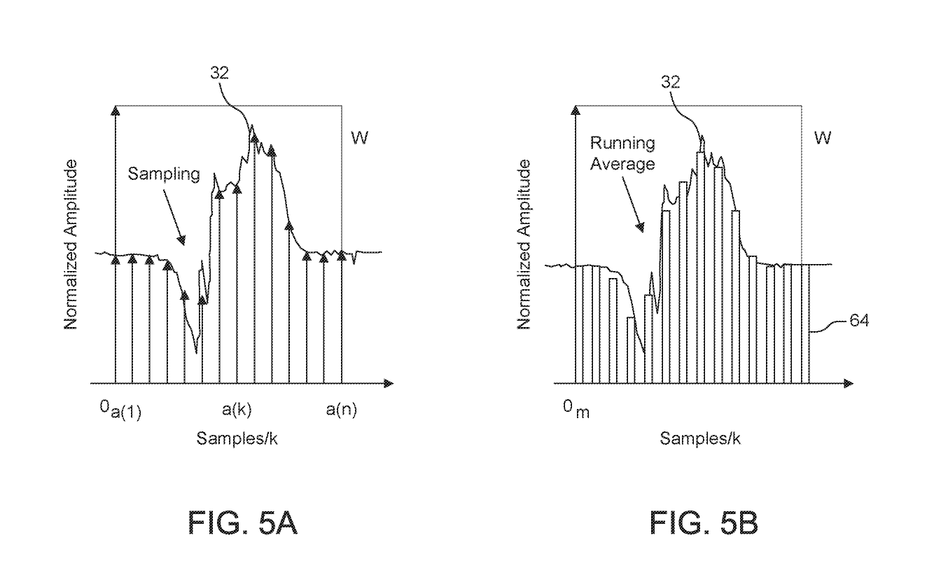

FIGS. 5A and 5B are normalized amplitude plots that illustrate how artificial intelligence (AI) applications executing on the mobile user devices extract feature vectors from the raw sensor data. FIG. 5A shows how the mobile AI applications might sample the raw sensor data in the time domain to extract feature vectors with sample size=n, window size=W. FIG. 5B shows the extracted features after smoothing using median filtering. Typically, different signal preprocessing with different filters are done based on the sensors being used and the sensitivity/accuracy desired.

In the illustrated example, FIGS. 5A and 5B show preprocessing of time-domain features on the user devices. The system 200 uses the innovative preprocessing stage 10 in FIG. 4, for processing the sensor signals in the form of sensor data 32 on each user device 20. In order to elucidate a basis set of vectors from the signals from multiple sensors, part of the preprocessing stage 10 involves not only signal preprocessing but also uses trained neural networks. These neural networks analyze the signals, remove the noise and output the basis set of signals that make up the input signals.

To obtain the basis set of vectors, the AI application 14 needs to learn the time and space correlations of the sensor outputs. In one example, the system learns the difference between individuals drinking from a cup, when the individuals have and do not have hand tremors. The speed and overall movements in these two cases will have different responses. After learning, if needed, the same signal can be reconstructed as a simulation. This is typically performed in the command and control module 142, where extensive simulation generates more data and training, and this is them pushed back to the user devices 20.

If there is power left in the user device 20, the user device 20 will use the trained model 70 sent from the command and control module 142 and preprocess (new) raw sensor data 32 for learning. Alternatively, the user device 20 can send the sensor data 32 to the command and control module 142 to perform large scale learning and revalidation tests.

Before using the raw sensor data 32 from any sensor 60, the signal is normalized based on sensor parameters and sensitivity.

Using lossless compression the signal that is now digitized is tagged with a unique id and sensor type and saved.

In general, learning is performed at the command and control module 142. As a result of the learning, the command and control module 142 creates a trained model 70 and sends the trained model 70 for deployment on the user devices 20. In rare cases, the learning is performed on the user devices 20 if the connection between the local software 89 and the command and control module 142 is lost or otherwise unavailable, but only after an initial trained model 70 is deployed by the command and control module 142 on the user devices 20.

During learning, principal component analysis (PCA) of the complete eigenset (eigenvectors) is done on the stored signals from each sensor for a particular behavior or feature vector. This forms the basis set. PCA now forms the basis for modeling and simulations.

The signal typically will have noise (random and white noise) and also is error-prone and effected by environmental changes. Therefore, the processing of time-domain feature extraction of the signal begins with medial filtering. Signal is intercepted by window W based on threshold judgment. The running average is from N samples, and detection time window is T. Further assuming the sensor signal is a stochastic variable, periodic evaluations of the following correlations will take place: correlations between signals from the same sensor type from different individuals at different times as well as time correlations between signals from the same phone and the same sensor at different times.

Note that the data collected by the sensor sampled from raw signals is discrete in the time domain.

The correlations found between sensor signals from the same sensors are also calculated periodically and saved and are continuously used during training of the trained neural network to be deployed on each user device.

It is also used by the trained neural network on each user device to detect new learning and for unsupervised learning after deployment.

The time shift used to transform signals and calculate correlations is critical. Extensive simulations of sensor signals from a given basis set will help determine its value from a given sensor. Typically it is some multiple of the correlation time for a signal that repeats itself for a given behavior or feature vector.

T varies and is dependent on the power usage of the phone as the control software changes sampling rate to control the application power usage.

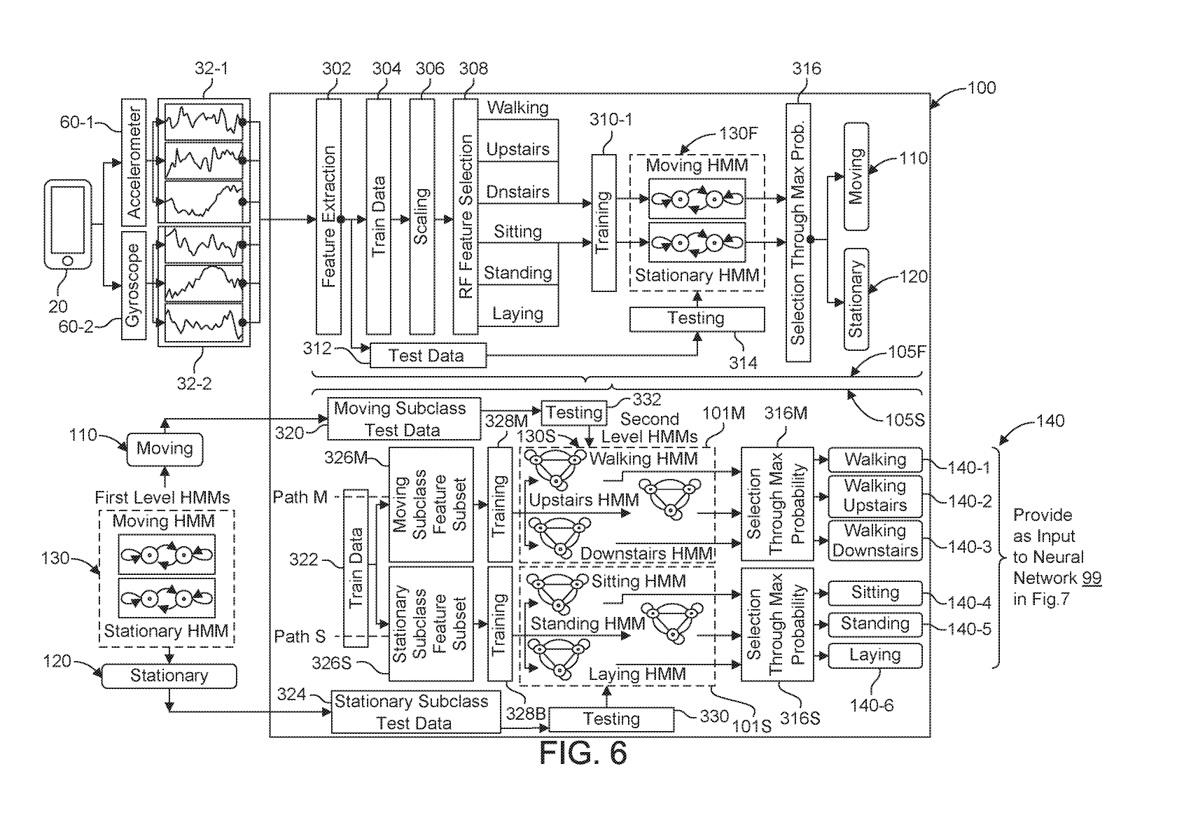

FIG. 6 shows more detail for a first level neural network 105F and a second level neural network 105S within the PCA system 100.

The neural networks 105 are based upon continuous HMMs classification and analysis of sensor data 32 of cellular phone data of gyroscopes and accelerometers for training and/or testing. The figure shows flow of information, feature selection and extraction, and assignment of probability to “context signals” (moving context signals 110 and stationary context signals 120) in the first level, followed by further classification of the context signals into “conduct signals” 140 in the second level.

The first level neural network 105F includes first level HMMs 130F and various other components/modules.

The second level neural network 105S includes second level HMMs 130S and various other components/modules.

The trained neural network on each mobile user device is implemented using a hybrid analog recurrent neural network (a-RNN) with long short term memory (LSTM) and a hidden Markov model (HMM) based neural network.

For a given instance of sensor data 32, PCA (Principal Component Analysis) is performed by the PCA system 100 of the HPC cluster 80. Then, a complete set of possible signals using the basis vectors is found. This along with the experimental data collected forms the training, testing and validation data sets. Included here is also the correlation coefficients calculated for the simulated and experimental signals.

This hybrid neural network-based PCA system 100 has a continuous HMM at two levels, and an RNN that is designed to handle a continuous time series of signals. After the neural networks within the PCA system 100 are trained, they will proceed in an unsupervised manner. In examples, the RNN might include a LSTM (long short term memory) neural network or a Convolutional Neural Network (CNN). RNNs are often used with time-sequenced, highly correlated non-linear data.

In general, the first level neural network 105F is based on continuous stationary HMMs (hidden Markov models) as the first level HMMs 130F for coarse classification of user device data of gyroscopes and accelerometers for training and/or testing. The figure shows flow of information through the first level HMMs 130F, feature selection and extraction, and assignment of probability to stationary or moving states, i.e., “context signals” (110 for moving context signals and 120 for stationary context signals).

The first level neural network 105F has the following components and generally operates as follows. Sensor data 32-1 from accelerometer sensor 60-1 and sensor data 32-2 from gyroscope sensor 60-2 is provided as input to a feature extraction module 302. The feature extraction module 302 extracts features from the input sensor data 32, and provides the extracted features as input to a train data module 304 and to a test data module 312.

At the train data module 304, the features are trained, and then scaled at scaling module 306. Upon completion of scaling, an RF Feature selection module 308 makes a determination as to various motion states of the user/individual, such as walking in general, walking upstairs or downstairs, sitting, standing, or laying down, in examples. These motion states are then provided as input to training module 310-1.

The first level HMMs 130F include a moving HMM and a stationary HMM that can identify whether the motion state sent by the training module 310-1 is moving or stationary, respectively.

At the test data module 312, test data is provided to a testing module 314.

The first level HMMs 130F then receive the motion state sent by the training module 310-1 and the test data from the testing module 314.

In response to the information sent by the training module 310-1 (and possibly the testing module 314), the first level HMMs 130F generate output to a selection through maximum probability module 316. This probability module 316 determines whether the motion state is most likely moving or stationary, and produces moving context signals 110 or stationary context signals 120 in response.

The second level neural network 105S has the following components and generally operates as follows.

In the second level neural network 105S, further classification of the two context signals (moving 110 or stationary 120) into “conduct signals” 140 is performed, also using neural network based HMMs as the second level HMMs 130S.

The moving context signal 110 is divided into subclasses of normal walking, walking downstairs or upstairs. The stationary context signal 120 is further divided into subclasses of sitting, standing or lying down.

The second level HMMs 130S include a moving portion 101M and a stationary portion 101S. The moving portion 101M includes separate HMMs for each of the following motion states: walking, upstairs, and downstairs. The stationary portion 101S includes separate HMMs for each of the following motion states: sitting, standing, and lying down.

The conduct signals 140 include movement-based conduct signals and stationary-based conduct signals. The movement-based conduct signals include walking 140-1, walking upstairs 140-2, and walking downstairs 140-3. The stationary-based conduct signals include sitting 140-4, standing 140-5, and laying 140-6.

In the illustrated example, the moving context signals 110 are provided as input to a moving subclass test data module 320, and the stationary context signals 120 are provided as input to a stationary subclass test data module 324. The test data from the moving subclass test data module 320 is sent to a testing module 332, and the test data from the stationary subclass data module 324 is sent to a separate testing module 330.

At the same time, a training data module 322 executes two parallel paths M and S for the second level HMMs 130S. The training data module 322 represents the state of the second level neural network 105S during training. In Path M, a moving subclass feature subset module 326M receives the trained data, and sends feature subset output to a training module 328M. The training module 328M then trains the moving portion 101M of the second level HMMs 130S to further determine whether the user is walking in general, walking downstairs or upstairs. Then, the output of these HMMs is provided as input to a selection through maximum probability module 316M. This probability module 316M determines whether the output of the moving portion 101M of the second level HMMs 130S is one of the movement-based conduct signals: walking 140-1, walking upstairs 140-2, and walking downstairs 140-3.

In a similar vein, in Path B, a stationary subclass feature subset module 326S receives the trained data, and sends feature subset output to a training module 328S. The training module 328S then trains the stationary portion 101S of the second level HMMs 130S to further determine whether the user is sitting, standing or laying down. Then, the output of theses HMMs is provided as input to a selection through maximum probability module 316S. This probability module 316S determines whether the output of the stationary portion 101S of the second level HMMs 130S is one of the stationary-based conduct signals: sitting 140-4, standing 140-5, and laying 140-6.

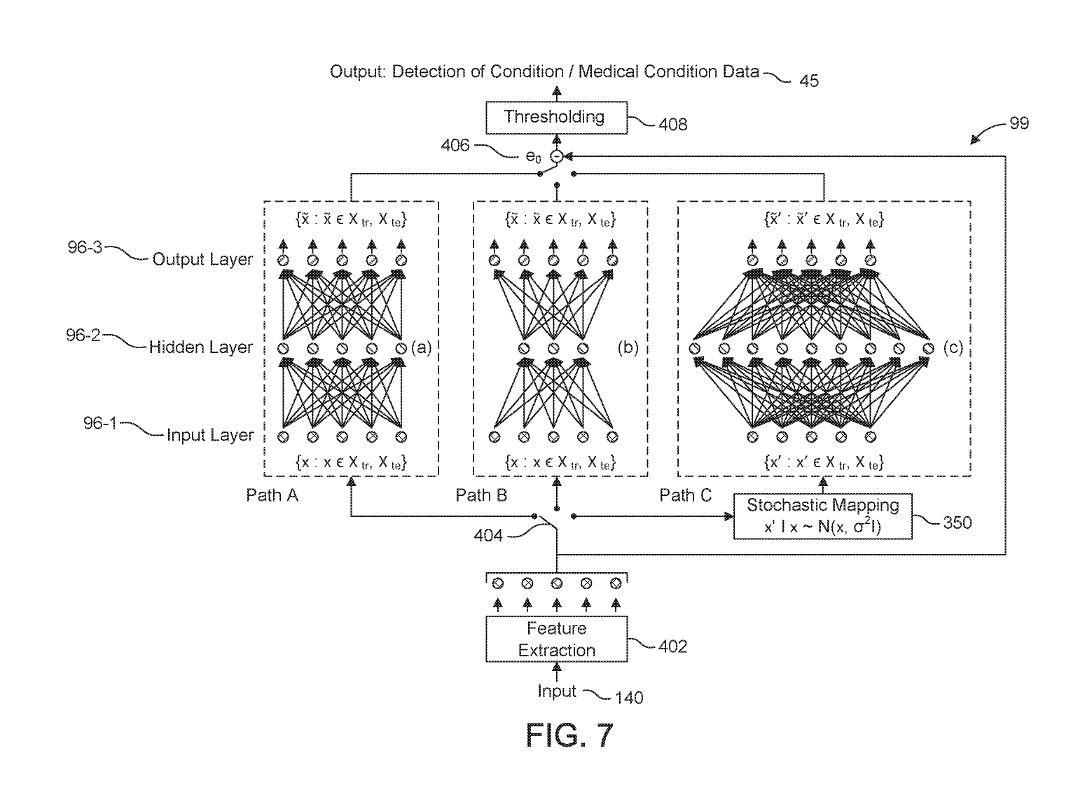

The conduct signals 140 are then provided as input to the neural network 99 of the user device 22 in FIG. 7.

FIG. 7 shows a neural network 99 at the user device 20. The neural network 99 extracts features from input information, where the input is the HMM stage outputs (i.e. the conduct signals 140) of the PCA 100 in FIG. 6.

The neural network 99 is formed from three layers: an input layer 96-1, a hidden layer 96-2, and an outer layer 96-3.

In general, the conduct signals 140 are provided as input to a feature extraction module 402, which extracts features. A switch 404 controls whether the features are sent along one of three paths A, B, and C through the layers 96 and towards a reconstruction error module 406. The extracted features are also sent to the reconstruction error module 406.

If the switch 404 is selected for path C, the features are first sent to a stochastic mapping module 350 prior to being sent through the layers 96.

A reconstruction error module 406 determines reconstruction error 352 between the inputs. The reconstructed features are then processed by a thresholding module 408 which finally detects the condition. The figure shows the a-RNN based neural network 99, which after training uses the final thresholding step 408 before finding the one or more conditions. The conditions then form the medical condition data 45.

Once the HMM of the neural network 99 is deployed on the user device 20, the HMM can identify new training in which case this can be used to retrain and deploy via a build in unsupervised loop that creates new HMM states on the fly. The global database 46 is updated, and learning from one user device 20 can be globally applied to update to the entire deployment of user devices 20 via a software update. Signals are now annotated in the database 46 and a semi-basis set is available. In more detail, the user devices 20 extract the basis datasets and mark them as being in such a semi-basis state until verified by the command and control module 142.

The RNN trained and deployed has more than two hidden layers and is thus a deep neural network, and incorporates LSTM (long short term memory) units.

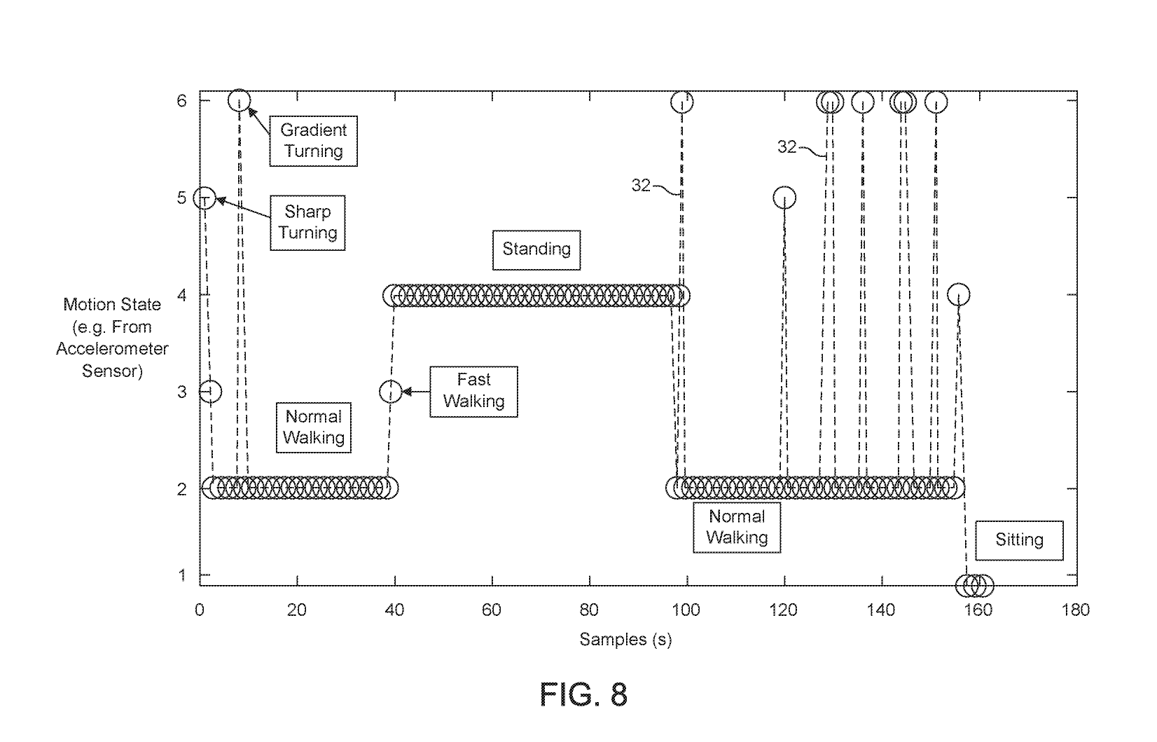

FIG. 8 is a plot of device sensor data 32 over time. By tracking this data, the user's motion state is resolved and determined. The motion state determination is a precursor to condition determination.

In the illustrated example, the motion states include normal walking, sharp turning, gradient turning, standing, fast walking, normal walking, and sitting are shown.

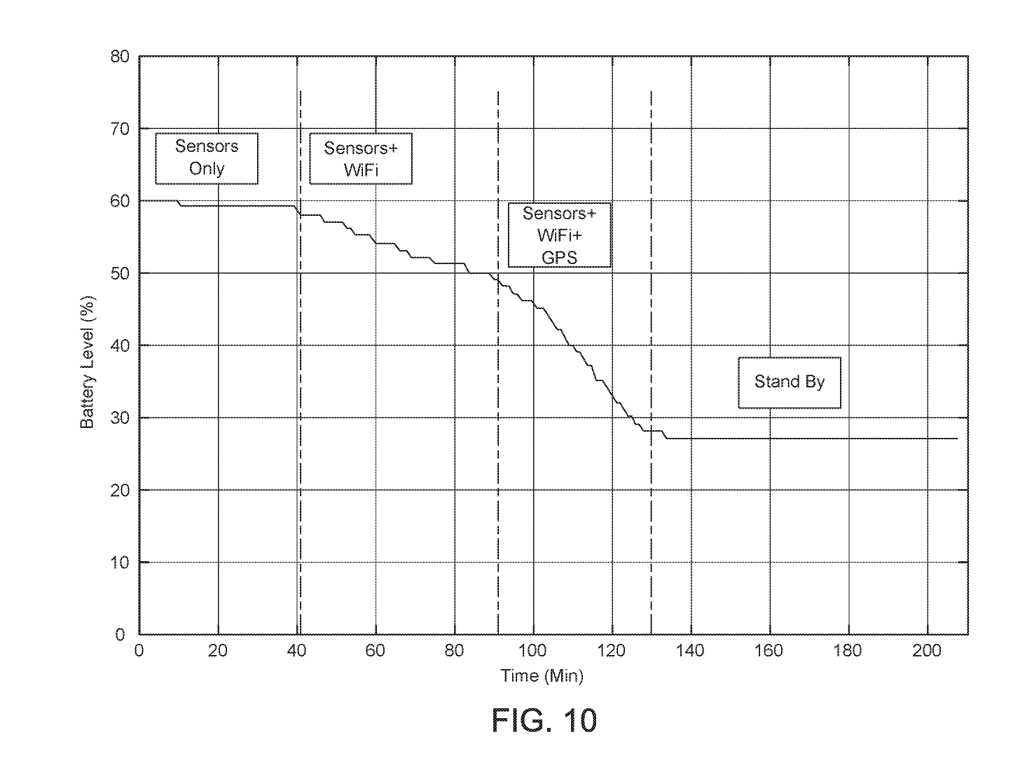

FIG. 9 shows operation of the differential update system 500 for two-way data transfer between the user devices 20 and the distributed computer network 16. As described hereinbelow, in FIG. 10, the user device power usage is small. This and the fact that new learning or a diagnosed condition, if detected, is uploaded via a differential update from the local sparse database 26 to the controller 44 (and database 46) to yield the longest metric for correlated signals, thus maximizing battery life and ensuring data processing from the sensors 60 occurs in a continuous manner.

Using variable sampling rates, instead of updating constantly, results in considerable power savings. Using differential updates from the sparse databases 26 of and intelligent analysis of signals in the user devices before uploading results in less bandwidth requirements and significant power saving.

With differential updates of the sparse data 26 from each user device 20, and software updates from the controller 44 to the local AI application 14 on each user device with new learning, each user device 20 is expected to have about 20 MB (upper limit) downloads, and about 5 MB (upper limit) uploads. The controller 44 (specifically, the command and control module 142 executing on the controller 44) must have access to (assuming 100,000 user devices are deployed) about 2.5 TB of data in the cloud, in one example.

The sparse database 26 on each user device 20 also is used by the controller 44 for updates of information needed for preprocessing based on new learning or in storing information from unsupervised learning on the user device after deployment.

The (learnt) signals on each user device form a unique sparse local dataset (local database 26). The learning stage is parallelized across the user device 20 of each participant. The difference (“diff” in the figure) between the initial downloaded model and the current updated model is compressed and uploaded to the cloud database 46 where it is reconstituted.

Each local sparse data set 26 is selectively updated to a global hybrid cloud. The global database 46 is controlled by the controller 44. The controller 44 also engages in collecting and processing sparse database 26 information periodically from each user device 20, reconstituting the models and using the information for training and identifying the basis set including associated feature vectors, new learning, pushing new learning via software updates to the user device, as well as being a supervisory and collaborating platform with high availability for controlling the deployment. This global platform also supports machine learning (ML) on the cloud database 46.

Updates to and from the distributed computer network 16 and mirrors 16M are done selectively and optimally to minimize streaming data traffic and to ensure scalability with respect to number of user devices 20 deployed. The distributed computer network 16 can be mirrored 16M to ensure the updates are as local to the deployment as possible. It is even possible to have the deployment widely spaced with each instance of the nearest hybrid cloud being updated asynchronously and the data being lazily synced on the mirrored global databases. The global database instances also can scale up and rebalance as needed.

The mobile AI application 14 that implements neural network operation on the user devices 20 has low power consumption, as compared to the power consumption of most benchmark applications on commonly available user devices. Examples of commonly available user devices 20 include: an Openmoko Neo Freerunner user device, revision A6, also known as Freerunner; an HTC Dream G1 user device, also known as G1; and a Google Nexus One user device, also known as H1. Examples of benchmark applications include a Suspend, Idle, Phone call, email (cell), email (wifi), web (cell), web (wifi), network (cell), network (wifi), video, and audio applications.

By way of comparison, the average power consumed for the Freerunner/G1/H1 devices for the aforementioned benchmark applications is as follows (in average mW):

Suspend: Freerunner (103.2), G1 (26.6), N1 (24.9)

Idle: Freerunner (333.7), G1 (161.2), N1 (24.9)

Phone call: Freerunner (1135.4), G1 (822.4), N1 (746.8)

Email (cell): Freerunner (690.7), G1 (599.4), N1 (n/a)

Email (wifi): Freerunner (505.6), G1 (349.2), N1 (n/a)

Web (cell): Freerunner (500), G1 (430.4), N1 (538.0)

Web (wifi): Freerunner (430.4), G1 (270.6), N1 (412.2)

Network (cell): Freerunner (929.7), G1 (1016.4), N1 (825.9)

Network (wifi): Freerunner (1053.7), G1 (1355.8), N1 (884.1)

Video: Freerunner (558.8), G1 (568.3), N1 (526.3)

Audio: Freerunner (419.0), G1 (459.7), N1 (322.4)

Experimentation has shown that that the AI application 14 has a low average power consumption compared to these benchmark applications. The average power consumption of the AI application 14 is similar to that of the audio playback applications on the Freerunner/G1/H1 user devices, in one example, minus the energy used by the speaker.

The control software/AI application 14 that will be deployed on each user device 20 controls the way differential updates occur. The local AI application 14 (control software) monitors and controls its energy usage automatically by changing sampling rates and other parameters to ensure no more than 5% power usage occurs in the user device 20. The AI application 14 also ensures that the differential updates provide an acceptable data rate for both training and characterization of conditions.

FIG. 10 shows the decrease in battery level with various operating modes on a Samsung Galaxy 2 user device 20. After 40 minutes with only sensors operating, the power level decreases by a mere 1%. But during the same time span, if WiFi is tuned on along with the sensors 32, the battery level decreases by slightly more than 7%. If additionally the GPS is turned on, the power level decreases by 20%. This illustrates that for a high value of the QoS (Quality of Service) metric it is important to minimize WiFi connection of the user device 20 to the cloud.

The system's innovative architecture minimizes the energy expended for communications by minimizing the amount of data transmitted while maximizing the information content. Through the use of Machine Learning algorithms, the system 200 can achieve vast data reduction without compromising the embedded latent health information signals in the AI application 14.

Existing user devices perform raw data collection at constant sample rates and without the unsupervised learning provided by the AI applications 14 on the deployed user devices 20 in the system 200. As a result, existing user devices cannot achieve the accuracy, scalability and QoS when compared to this inventive system 200.

The system 200 has intelligence and autonomy built in. In examples, the system 200 can vary sampling rates, move computation to the command and control module 142, and perform diff updates.

In general, the system 200 has access to a very large data set via the command and control module 142. The data set includes information sent from many user devices 20, and simulated data and preprocessed data from the command and control module 142. Thus, in one example, battery drain on each user device 20 can be controlled by limiting sampling and local processing at the user devices 20, and instead performing most of the computational sampling load at the command and control module 142. Such a capability enables improved accuracy and processing throughput at the user devices of the system 200 as compared to existing user devices.

The AI application 14 will automatically analyze and adjust sampling frequency and signal characterization so that differential updates are triggered at the right time with data that has high confidence value. Thus, new learning which occurs during the training phase and continues after deployment unsupervised, is accurately captured. Additionally, data that is meaningful in determining conditions is also uploaded to the cloud.

The interval of collection of signals/sensor data 32 from the various sensors 20 is adjusted by the AI application 14, based on QoS and energy analysis. However, as the interval collection varies so does the data rate. With time, the raw signals are aggregated and averaged before being saved. This is generally performed dynamically and typically in memory. For example, if the collection rate of signals is high (interval between collection of two signals low), more signals are averaged, and vice-versa. Thus the average rate of data 32 from a sensor 20 can be controlled. No more than 100 MB per day of raw and sparse processed data generation from each user device 20 will occur using this control technique and the local AI application 14 on each user device will use no more than 200 MB of local storage.

Generally, the raw signals are aggregated and averaged in memory 88 at the user devices 20 for the following reasons. Performing these operations in-memory (rather than the local software 89 using CPU resources) is faster, reduces power consumption, and limits the number of time-consuming floating point operations per second (FLOPS) that would otherwise be performed by the CPU 82.

Also, the level at which the raw signals are aggregated and averaged in memory 88 versus using CPU resources is dependent upon the amount of sensor data 32 at the user devices 20. When the amount of sensor data 32 increases above a threshold amount at the user devices 20, the sensor data 32 can be captured and preprocessed in the memory 88, thus limiting write operations on the user devices 20. Dynamically the memory 88 is flushed by the local software 89 after aggregation. Weak and noisy signals of the sensor data 32 are also reinforced this way.

Such a QoS implementation allows for down-sampling or up-sampling of the sensor data 32 from the sensors 60 dynamically. This is an innovative implementation in the preprocessing stage.

FIGS. 11 and 12 show different operational models for how the sensor data 32 at each of the user devices 20 might be fused at the user devices 20 by the AI application 14 before sending the sensor data 32 to the command and control module 142.

The AI application 14 on each user device 20 decides on which of these models to use, and can change this in real time based on power requirements, sensitivity required and availability/accessibility of the sensors 60, in examples. The AI application 14 performs extended simulations and training to predict fusion methods to use in addition to other learning described and updates the AI applications with new learning.

After the AI application 14 fuses the sensor data 32 from one or more sensors 60, the AI application 14 sends the fused sensor data to the command and control module 142. The command and control module 142 uses the fused sensor data and additional simulations and training to produce a new trained neural network model 70. The command and control module 142 then sends the trained model 70 for deployment on the user device 20.

FIG. 11 shows a first fusion model of the AI application 14 for an exemplary user device 20. Here, the sensor data 32 from multiple IMUs/sensors 60-1 through 60-N are first combined/fused, and then formatted. Here, the sensor data 32 are fused into a collection of statistical signals/weights that represent the “raw signals” of the sensor data. Typical fusion algorithms/methods are shown in the figure.

In the illustrated example, several types of filters can be used and at several locations in the case of sensor fusion. The filters include Kalman filter, extended Kalman filter, an unscented Kalman filter, in examples. Based on the sensor data 32 from the sensors 60 being fused, the local software 89 tries several approaches in parallel with the command and control software 142. The command and control software 142 uses the continuously available HPC resources 80 and then decides on the best method to use.

In more detail, at the AI application 14, an IMU Observation Fusion module 502 receives sensor data 32 from the multiple sensors 60-1 . . . 60-N. The fusion module 502 provides the combined sensor data 32 to an INS Kalman filter module 504.

The Kalman filter module 504 also accepts sensor data 32 from the GPS sensor 60. The filter module 504 also has a system feedback loop 510.

The Kalman filter module 504 has the following inputs. The inputs include the sensor data 32 from the GPS sensor 60, the feedback loop 510, and the combined sensor data 32 from the fusion module 302.

The Kalman filter module 504 filters its input to provide filtered sensor data 20 as its output. This filtered sensor data is sent to a final output buffer 506. The AI application 14 can then transmit the filtered sensor data 20 from its final output buffer 506 over a communications network (e.g. cellular) for further analysis by the PCA system 100.

The “fused” version of the sensor data 32 at the final output stage 506 includes statistical versions of the sensor data 32. These statistical versions include: GPS signals, heart rate signals, blood flow signals, accelerometer signals, and gyroscope signals, in examples. In other examples, the signals can be from the following devices: a Geiger counter/dosimeter, a chemical or bio detector, an acoustic sonar device, a LIDAR device, a blood sugar monitor, and from a pacemaker or brain electrode.

FIG. 12 shows how the sensor data 32 from each sensor 60 including inertial measurement systems (IMUs), can alternatively be pre-processed and then fused by the AI application 14 on each user device 20, prior to sending the sensor data 32 for analysis by the PCA system 100.

In more detail, sensors 60-1 . . . 60-N on user device 20 send their sensor data 32 for pre-processing by the AI application 14. At the AI application 14, separate local INS filter modules 570-1 . . . 570-N for each sensor 60-1 . . . 60-N receive the sensor data 32 from each sensor 60. A GPS observations/SINS Solutions module 572 also provides input to each of the local INS filter modules 570.

Alternatively, rather than fusing the sensor data 32 locally at each user device 20, the sensor data 32 can be sent to the command and control module 142 of the global software 79. At the controller 44, the command and control module 142 uses the sensor data 32 sent by one or more user devices 20 in conjunction with additional simulations and training, and sends a trained model 70 for use at the user devices 20 in response.

The local INS filter modules 570 then provide filtered versions of the combined sensor data 32/GPS observations as input to a master fusion module 574. The master fusion module 574 combines its input to produce a fused and filtered version of the sensor data 32. This sensor data 32 is then sent to final output buffer 506.

Multiple sensors 60 in different and dynamic changing configurations can now be used. These configurations are limited only by computation, power, weight and design considerations.

The system 200 can use any external sensors if needed. These sensors 60 may be accessible by a USB port of the user device 20 or by Bluetooth or other network protocols. The additional sensors 60 could be used for the following purposes: to increase the accuracy of the inbuilt sensors—accelerometer/gyroscope/compass; and/or to add functionality like a wrist watch that monitors heart rate using photoplethysmography, or that monitors blood pressure using an arterial pulsimeter, in examples.

While this invention has been particularly shown and described with references to preferred embodiments thereof, it will be understood by those skilled in the art that various changes in form and details may be made therein without departing from the scope of the invention encompassed by the appended claims.

What is claimed is:

1. A distributed analytics system for identification, determination and early warning of disease and/or injuries, the system comprising:
mobile computing devices carried by users that monitor the users via sensors; and
a distributed computer network that communicates with the mobile computing devices for facilitating the identification, determination and early warning of disease and/or injuries in the users.

2. The system of claim 1, wherein the mobile computing devices include computer processing units executing artificial intelligence (AI) applications and sensors that provide sensor data to the AI applications.

3. The system of claim 2, wherein the AI applications executing on the computer processing units include neural networks.

4. The system of claim 3, wherein the AI applications create training datasets that include the sensor data and send the training datasets for processing by a principal component analysis system of the distributed computer network that determines principal components.

5. The system of claim 4, wherein the principal components include feature vectors, basis vectors, and eigenvectors that are used by a training system to create model training data for a neural network model for the neural networks executing on the computer processing units of the mobile computing devices.

6. The system of claim 3, wherein new sensor data from the sensors are used to train and/or update the neural networks of the devices.

7. The system of claim 3, wherein the mobile computing devices locally update the neural networks when communications between the distributed computer network and the devices are lost.

8. The system of claim 1, wherein data connections between the mobile computing devices and the distributed computer network are created dynamically by the mobile computing devices including peer-to-peer ad hoc connections to other mobile computing devices.

9. The system of claim 1, wherein the distributed computer network comprises a differential update system, a diagnosis system, and a training system.

10. The system of claim 9, wherein the diagnosis system creates medical condition reports for individuals carrying the mobile computing devices.

11. The system of claim 9, wherein the diagnosis system creates medical condition reports for public health officials and/or to medical companies performing medical device and medication trials and patient monitoring.

12. A method for identification, determination and early warning of disease and/or injuries, the method comprising:
monitoring users with mobile computing devices carried by the users; and
a distributed computer network communicating with the mobile computing devices for facilitating the identification, determination, and early warning of disease and/or injuries in the users.

13. The method of claim 12, further comprising the mobile computing devices executing artificial intelligence (AI) applications that process sensor data collected by sensors of the mobile computing devices.

14. The method of claim 13, wherein the AI applications executed by the mobile computing devices include neural networks.

15. The method of claim 14, further comprising the AI applications creating training datasets that include the sensor data and send the training datasets for processing by a principal component analysis system of the distributed computer network that determines principal components.

16. The method of claim 15, wherein the principal components include feature vectors, basis vectors, and eigenvectors, the method further comprising a training system using the feature vectors, basis vectors, and eigenvectors to create model training data for a neural network model for the neural networks executing on the mobile computing devices.

17. The method of claim 14, further comprising training and/or updating the neural networks of the devices using new sensor data from the sensors.

18. The method of claim 14, further comprising the mobile computing devices locally updating the neural networks when communications between the distributed computer network and the devices are lost.

19. The method of claim 12, further comprising dynamically creating data connections between the mobile computing devices and the distributed computer network, which connections include peer-to-peer ad hoc connections to other mobile computing devices.

20. The method of claim 12, further comprising a diagnosis system of the distributed computer network creating medical condition reports for individuals carrying the mobile computing devices.

21. The system of claim 20, wherein the diagnosis system creates medical condition reports for public health officials and/or to medical companies performing medical device and medication trials and patient monitoring.

22. A distributed analytics system, comprising:
a command and control software module that deploys neural network models to user devices and updates the neural network models in response to sensor data sent from sensors of the mobile user devices; and
one or more networks that provide communications between the command and control module and the mobile user devices.
